(19) United States
(12) Patent Application Publication
(10) Pub. No.: US 2014/0213537 A1
(43) Pub. Date: Jul. 31, 2014

(54) RESVERATROL EXTRACTION FROM GNETUM AFRICANUM

(71) Applicants: Carole Robert, Blainville (CA); Sue Wehner, Ste-Marthe (CA)

(72) Inventors: Carole Robert, Blainville (CA); Sue Wehner, Ste-Marthe (CA)

(73) Assignee: PHARMAFRI-CAN INC., Levis, QC (CA)

(21) Appl. No.: 13/812,450

(22) PCT Filed: Jul. 26, 2011
(86) PCT No.: PCT/CA2011/000872

§ 371 Date: Feb. 5, 2014

Related U.S. Application Data
(60) Provisional application No. 61/367,579, filed on Jul. 26, 2010.

(51) Int. Cl. A61K 31/05; A61K 31/7032; A23L 2/52; A61Q 19/00; A61K 36/17; A23L 1/30; A61K 8/34; A61K 8/60

(52) U.S. Cl. CPC A61K 31/05; A61K 8/347; A61K 31/7032; A61K 8/602; A61Q 19/00; A61K 36/17; A23L 1/3002; A23L 2/52
USPC 514/25; 514/733; 206/461

(57) ABSTRACT

The invention relates to extracts and compositions from Gnetum africanum. It also concerns plant extracts enriched in resveratrol and methods for obtaining the same. Described herein are processed plant materials which comprise about 20 μg resveratrol per gram of dried plant material and plant extracts which comprise at least about 0.002% w/w resveratrol. Also described are resveratrol glycosides and compositions comprising same. The invention further encompasses food products, nutraceutical products, pharmaceutical compositions which comprise processed plant materials, plant extracts and/or resveratrol glycosides.

### FIELD OF THE INVENTION

The invention relates to the field of natural products. More particularly, it concerns extracts and compositions from Gnetum africanum. It also concerns plant extracts enriched in resveratrol and methods for obtaining the same.

### BACKGROUND OF THE INVENTION

Resveratrol is a compound having the chemical formula C₁₄H₁₂O₃ and it is represented by the following structure:

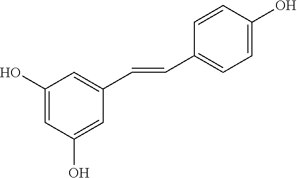 [SMILES: Oc1ccc(/C=C/c2cc(O)cc(O)c2)cc1]

Resveratrol is produced naturally by several plants and can be synthesized chemically. Plant extracts obtained so far only contain low concentrations of resveratrol. For instance, resveratrol is found in the skin of red grapes (at a concentration of about 10 μg/g) and it is a constituent of red wine (at a concentration of about 13 μg/ml).

Resveratrol has attracted considerable attention since anti-cancer, anti-inflammatory, blood-sugar-lowering and numerous beneficial cardiovascular effects have been attributed to it. However, experiments have shown that resveratrol alone, even at a relatively high dose, does not yield significant clinical effects in man. Therefore it is becoming accepted that the beneficial effects of resveratrol rely on the presence of one or more unknown compounds. Thus, there is a need for compositions and extracts enriched with high concentrations of resveratrol derived from plants.

Gnetum africanum is a popular vegetable commonly found in tropical Africa. It is collected in the wild (rather than cultivated) and it is added to foods and made into fusions. Although traditional African medicine includes a few medical uses for Gnetum africanum, there is no clear scientific evidence of any therapeutic efficacy for that plant. Furthermore, the conditions under which it would be possible to obtain biologically active extracts or products derived from that plant have not been properly documented.

Therefore, there is also a need for plant extracts, particularly extracts from Gnetum africanum comprising resveratrol, and methods for extracting resveratrol. There is also a need for pharmaceutical compositions comprising such plant extracts and resveratrol, as well as nutraceuticals and cosmetics comprising same.

The present invention addresses these needs and other needs as will be apparent from review of the disclosure, figures and description of the features of the invention hereinafter.

### BRIEF SUMMARY OF THE INVENTION

The present inventors have found a new vegetal source from which it is possible to obtain plant material and extracts more concentrated in resveratrol than any previously known vegetal source which provides only about 10-15 μg resveratrol per gram of plant material.

Accordingly, one aspect of the invention relates to processed plant material comprising about 20 μg resveratrol per gram of dried plant material. In one embodiment the plant material is derived from the plant Gnetum africanum, preferably the leaves or the stem.

Another aspect of the invention concerns a plant extract which comprises at least about 0.002% w/w resveratrol. In one embodiment the plant material is derived from the plant Gnetum africanum, preferably the leaves or the stem.

The invention further relates to purification methods for obtaining a plant extract concentrated in resveratrol. In one embodiment the method comprises:
a) providing plant material from Gnetum africanum;
b) submitting said plant material to a solvent extraction; and
c) obtaining a plant extract concentrated in resveratrol, said plant extract comprising at least about 0.002% w/w resveratrol.

The invention also concerns resveratrol glycosides and compositions comprising same. In one embodiment the resveratrol glycoside consists of a compound identifiable under detection conditions of Table 1 by the presence of a peak having a retention time of 9.1 min on a HPLC-APCI-MSD chromatogram. In another embodiment the resveratrol glycoside consists of a compound identifiable under detection conditions of Table 1 by the presence of a peak having a retention time of 9.5 min on a HPLC-APCI-MSD chromatogram.

The invention further relates to food products and nutraceutical products which comprise at least one of a processed plant material, a plant extract and a resveratrol glycoside as defined herein.

The invention also relates to cosmetic compositions which comprise at least one of a processed plant material, a plant extract and a resveratrol glycoside as defined herein in combination with a suitable cosmetic carrier, diluent or excipient.

The invention also relates to antioxidant compositions which comprise at least one of a processed plant material, a plant extract and a resveratrol glycoside as defined herein in combination in combination with a suitable carrier, diluent or excipient.

The invention also relates to pharmaceutical compositions which comprise at least one of a processed plant material, a plant extract and a resveratrol glycoside as defined herein in combination in combination with a pharmaceutically acceptable carrier, diluent or excipient.

Another related aspect of the invention concerns the use of a processed plant material, a plant extract and/or a resveratrol glycoside as defined herein for the manufacture of pharmaceutical compositions and drugs. One particular example is a pharmaceutical composition comprising a plant extract from Gnetum africanum. In embodiments, the extract comprises at least about 0.002% w/w resveratrol and/or one or more resveratrol glycosides.

Accordingly, in some aspects the processed plant material, a plant extract and/or a resveratrol glycoside according to the invention are used for the manufacture of medicaments, and more particularly medicaments useful in the prevention and/or treatment of skin diseases, cardiovascular diseases, inflammatory diseases, blood lipid abnormalities and pain.

An advantage of the present invention is that it provides a new natural source of resveratrol. It also provides plant extracts rich in resveratrol and other components. The invention further provides compositions comprising extracts from Gnetum africanum which may have numerous health, nutritional and cosmetic benefits.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

FIG. 1 is a line graph showing a HPLC-DAD profile of extract #E-01 (leaves from the Equatorial forest) obtained at the monitoring wavelength of 325 nm. Resveratrol is the major peak.

FIG. 2 is a line graph showing an ion chromatogram of HPLC-APCI-MSD profile of ethanolic extract of extract #E-47 (leaves from the Equatorial forest). Two overlaid lines are represented, one of 229 [M+H]⁺ and the other of 391 [M_{+ glyc]}⁺. In addition to resveratrol (peak #3), two glycosides (peak #1 and peak #2) are visible.

### DETAILED DESCRIPTION OF PREFERRED EMBODIMENTS

The present inventors have found a new natural source of resveratrol. More particularly the inventors have found that the plant Gnetum africanum can be a valuable source of resveratrol and they have obtained extracts from that plant containing resveratrol at much higher concentrations than that of any other plant extract produced so far.

As used herein, the term “resveratrol” refers to the compound having the chemical formula C₁₄H₁₂O₃ and the structure illustrated hereinbefore. Resveratrol is also defined by the CAS Registry Number 501-36-0 and known under other names, including, but not limited to, trans-resveratrol, trans-3,5,4′-Trihydroxystilbene; 3,4′,5-Stilbenetriol; (E)-5-(p-Hydroxystyryl)resorcinol; and (E)-5-(4-hydroxystyryl)benzene-1,3-diol.

### Plant Extracts

One aspect of the present invention relates to harvested plant material comprising about 20 μg, 30 μg, 40 μg, 50 μg, 75 μg, 100 μg or more resveratrol per gram of dried plant material. In various embodiments, the plant material is dried and comprises from at least about 25 μg/g, 50 μg/g, 75 μg/g, about 100 μg/g, about 250 μg/g, about 500 μg/g, about 1000 μg/g, about 1500 μg/g, about 2000 μg/g, about 2500 μg/g, about 3000 μg/g, about 4000 μg/g, about 5000 μg/g, about 10 000 μg/g, about 15 000 μg/g, or at least about 16 500 μg/g, resveratrol.

As used herein, the term “processed plant material” refers to one or more tissues of a plant which has been harvested for its known properties (e.g. resveratrol content) and prepared (e.g. washed, dried, ground, treated, refined, transformed, packaged, etc.) with the purpose of large scale commercialization. For the purpose of clarification, infusions and other similar concoctions of Gnetum prepared according to African traditional medicine are not considered a “process plant material” according to the invention, and as such, are exclude from the scope of the invention.

Another aspect concerns a plant extract comprising at least about 0.002% w/w, 0.003% w/w, 0.004% w/w, 0.005% w/w, 0.075% w/w, 0.01% w/w, or more resveratrol. In various embodiments, the plant material is dried and comprises from at least about 0.0025% w/w, 0.075% w/w, 0.01% w/w, 0.02% w/w, 0.05% w/w, 0.1% w/w, 0.5% w/w, 1% w/w or more resveratrol.

As used herein, the term “plant extract” refers to a concentrated preparation of a plant material obtained by isolating or purifying desired active constituents with one or more extraction techniques. Examples of extraction techniques include, but are not limited to, solvent extraction (e.g. ethanol, methanol, ethyl acetate), chromatography, and the like. For the purpose of clarification, infusions and other similar concoctions of Gnetum prepared according to African traditional medicine are not considered a “plant extract” according to the invention, and as such, are exclude from the scope of the invention.

Preferably the plant material and the plant extract derive from the plant Gnetum africanum. As used herein, the term “derives from” or “derived from” or refers to a product, compound or composition which is(are) obtained from, or can be traced back, to a given definite source. A particular example according to the invention includes resveratrol extracted or isolated from Gnetum africanum.

As used herein, the terms “Gnetum africanum”, “G. africanum” or “Gnetum” refers to the plant which commonly grows in tropical Africa. The plant is commonly called “mfumbwa” (kikongo), “longongia” (lingala) and “banvale” (Azande). Many different varieties are known, including the Asusutan, the Oron, the Ikom, the Welw and the Koko variety. Various sources of Gnetum africanum can be used according to the invention, including Gnetum from the Mayombe forest the equatorial forests of Congo. In preferred embodiments, the plant extracts according to the invention are obtained from the Gnetum africanum plants growing in the equatorial forests of Congo. Preferably, the leaves, stems and/or roots are used for obtaining a plant extract according to the invention but it is conceivable that other plant tissues such as flowers, fruits, tubers, corms, etc. may be also used. The invention further encompasses the plant Gnetum bucholzianum, and processed materials, extracts and resveratrol glycoside therefrom.

In preferred embodiments, it is the stem or leaves of Gnetum africanum, most preferably its stem, which is used for obtaining the plant material, plant extract(s), and resveratrol or a resveratrol glycoside according to the invention.

Those skilled in the art know how to identify useful sources of a plant material or plant extract and how to measure levels of resveratrol in different types of extracts or compositions. Exemplary methods for resveratrol quantification are provided herein in the Examples section. Similarly, Example 3 provides detailed analyses of the content of various biochemicals (e.g. lipids, proteins, sugars, etc.) in selected parts of Gnetum africanum. Those skilled in the art can thus refer to those analyses for comparison in assessing whether a plant material or plant extract is derived from Gnetum africanum.

The present inventors have further identified new resveratrol glycosides referred to herein as “resveratrol glycoside-1” and “resveratrol glycoside-2”. As described hereinafter at Example 1, and more particularly in Table 1, the resveratrol glycosides are identifiable as distinct peaks by HPLC-MSD. More particularly, on a HPLC-APCI-MSD chromatogram and under detection conditions indicated in Table 1, resveratrol glycoside-1 is identifiable by the presence of a peak having a retention time of 9.1 min, whereas resveratrol glycoside-2 is identifiable as a peak having a retention time of 9.5 min. For comparison, resveratrol has a retention time of 10.1 min. Table 1 further provides mass spectrometric fragmentation pattern for identification of resveratrol glycoside-1 and -2.

Accordingly, an additional aspect of the invention concerns resveratrol glycoside-1 and resveratrol glycoside-2 in an isolated or purified form. The resveratrol glycoside-1 and -2 are isolated or purified from a plant, preferably from Gnetum africanum. The invention further encompasses compositions (e.g. pharmaceutical compositions) comprising at least one of resveratrol glycoside-1 and resveratrol glycoside-2.

Another aspect of the invention further concerns a plant extract or a processed plant material, preferably from Gnetum africanum, the extract comprising resveratrol glycoside-1, resveratrol glycoside-2, or both. The plant extract may further comprise lipids (e.g. omega-6, omega-3, saturated and trans fats), minerals (e.g. Mg, Mn, Se, P, etc.), soluble and insoluble fibers, sugars (e.g. glucose), vitamins (e.g. vitamin E) and amino acids and proteins. Table 4 herein after provides a non limitative list of constituents which can be found in Gnetum africanum.

### Compositions and Uses

Another aspect of the invention concerns compositions and products comprising a plant material, a plant extract and/or a resveratrol glycoside as defined herein.

Typically the composition and products of the invention includes one or more active ingredients which are derived from Gnetum africanum, including but not limited to resveratrol, resveratrol glycolside-1, and resveratrol glycoside-2. In a preferred embodiment, the composition comprises at least 0.002% w/w resveratrol derived from Gnetum africanum. In various embodiments, the composition may comprise from at least 0.0025% w/w, 0.003% w/w, 0.004% w/w, 0.005% w/w, 0.075% w/w, 0.01% w/w, 0.02% w/w, 0.05% w/w, 0.1% w/w, 0.5% w/w, 1% w/w or more resveratrol.

Those skilled in the art will readily appreciate that plant materials, plant extracts and/or resveratrol glycosides according to the invention possess numerous beneficial properties. Accordingly, another related aspect of the invention concerns food products, food additives, nutraceuticals, cosmetics, pharmaceuticals and antioxidant compositions comprising a plant material, a plant extract and/or a resveratrol component, including the glycosides, as defined herein.

Examples of food products or food additives encompassed by the invention to which addition of a plant material, a plant extract and/or a resveratrol and/or resveratrol glycoside as defined herein may be beneficial, include but are not limited to dairy products such as yogurt, cheese and spreads etc., nutritional bars, herbal teas, various drinks, including fruit drinks and energy drinks.

Examples of nutraceuticals encompassed by the invention to which addition of a plant material, a plant extract and/or a resveratrol and/or a resveratrol glycoside as defined herein may be beneficial, include but are not limited to vitamins, nutritional supplements, sleep aids, memory aids.

Examples of cosmetic compositions encompassed by the invention to which addition of a plant material, a plant extract and/or a resveratrol and/or a resveratrol glycoside as defined herein may be beneficial, include but are not limited to facial creams, lotions, skin tonics, and the like.

Examples of pharmaceutical compositions encompassed by the invention to which addition of a plant material, a plant extract and/or a resveratrol and/or resveratrol glycoside as defined herein may be beneficial, include but are not limited to various types of cardiovascular drugs, lipid regulating drugs, anti-inflammatory drugs, analgesics, muscle-relaxants.

Antioxidant compositions encompassed by the invention to which addition of a plant material, a plant extract and/or a resveratrol glycoside as defined herein may beneficial, include but are not limited to antioxidant compositions useful for prevention of treatment of cardiovascular diseases and inflammatory diseases.

The invention further contemplates methods of use and methods of treatment, comprising administration of a plant material, a plant extract, a resveratrol, a resveratrol glycoside and/or composition as defined herein. More particularly, methods of treatment according to the invention comprise the administration to a subject in need thereof of an effective amount of such composition or extract, for cosmetic and/or for therapeutic purposes. Methods envisioned by the present invention include, but are not limited to, methods related to the skin (e.g. rejuvenating the skin, removing wrinkles, skin toners, skin softeners etc.), methods for preventing or treating a cardiovascular disease, methods for addressing blood lipids abnormalities and for controlling lipid levels in the blood (e.g. cholesterol, triglycerides, etc.), methods for treating inflammatory diseases or conditions, and methods for pain reduction.

Related aspect concerns the use of a plant material, a plant extract, a resveratrol, a resveratrol glycoside and/or a composition as defined herein for the manufacture of a medicament, nutraceutical or cosmetic product for use in these methods.

The plant material(s), plant extract(s), resveratrol, resveratrol glycoside(s) and/or composition(s) of the invention may be administered using different routes, for instance by the oral, intravenous (iv), intramuscular (im), depo-im, subcutaneous (sc), depo-sc, sublingually, intranasal, intrathecal, topical or rectal routes.

The compositions and extracts of the invention may be formulated under different suitable forms, such as dry powdered compositions, tablets or capsules, liquid solutions, suspensions, cream, ointment, lotion, paste, etc. The compositions and extracts according to the invention may also be marketed as a concentrate to be diluted by an end user.

The plant materials, plant extracts, resveratrol, resveratrol glycosides and/or compositions according to the invention may be prepared by various methods including but not limited to extracting, blending, grinding, homogenizing, suspending, dissolving, emulsifying, dispersing, and/or mixing selected extracts or ingredients derived therefrom, with selected excipient(s), carrier(s), adjuvant(s), diluent(s), or stabilizers. For preparing the composition of the invention, methods well known in the art may be used.

The compositions according to the invention may further comprise compounds and agents commonly used in food products or food additives, nutraceuticals, cosmetic compositions, pharmaceutical compositions and/or antioxidant compositions.

In addition, the plant materials, plant extracts, resveratrol, resveratrol glycosides and/or compositions of the invention may also contain additional ingredients, including but not limited to, metal chelators, metal scavengers, coating agents, preserving agents, solubilizing agents, stabilizing agents, wetting agents, emulsifiers, colorants (e.g. tracer dyes), flavours, odorants, salts, buffers, surfactants, solvents, coating agents and/or antioxidants.

The plant materials, plant extracts, resveratrol, resveratrol glycosides and/or compositions according to the invention may be packaged in different forms, which may include but are not limited to, sealed containers (e.g. a plastic or glass bottles, ampuls or vials), pouches (e.g. a bag or sachet), jars, tubes, blister packs, boxes, etc.

### Methods of Extraction

Those skilled in the art will readily appreciate that plant materials, plant extracts, resveratrol, resveratrol glycosides and/or compositions of the invention can be readily obtained by using various, processing, and/or purification methods and techniques. For instance, as exemplified hereinafter, samples comprising high levels of resveratrol can be obtained by using an ethanol extraction (see Example 1), a methanol extraction (Example 2), or an ethyl acetate extraction (Example 3) of plant materials from Gnetum africanum.

According to one particular aspect, the invention relates to a method for the preparation of a plant extract enriched or concentrated in resveratrol. In one embodiment the method comprises the steps of:
(i) providing plant material from Gnetum africanum;
(ii) submitting the plant material to a solvent extraction; and
(iii) obtaining a plant extract concentrated in resveratrol, the plant extract comprising at least about 0.002% w/w resveratrol.

In another embodiment the method comprises the steps of:
(i) grinding plant material (preferably previously dried) from Gnetum africanum;
(ii) extracting the ground plant material with one or more suitable solvents for obtaining a solvent extract;
(iii) recovering and drying the solvent extract to obtain the plant extract.

The methods may further comprise the steps of ultrasonicating the plant material in the presence of one or more suitable solvents at the solvent extraction step. In one embodiment, obtaining or recovering the resveratrol-concentrated plant extract comprises a centrifugation step. It may be preferable to carry out two rounds of (1) removing the supernatant and (2) re-suspending the pellet after each centrifugation step, and the supernatants from the rounds of centrifugation are pooled and dried.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further limiting.

### EXAMPLES

### Example 1

### Identification and Quantification of Resveratrol by HPLC-MS in Selected African Plants Following an Ethanol Extraction

### Experimental

Plant Material: The following samples of plant materials were received in dried unprocessed form: M02, M03, M21, M23, E01, E08, E46, E47. The samples are leaves collected from Gnetum africanum growing either in Mayombe forest (M) or the equatorial forest (E) of Congo. The leaves were left to dry before extraction and analysis.

Plant Extraction: Plants were ground with a coffee blender, and stored as a powder at −80° C. until used. Each plant material was weighed (1g) into a 50 ml centrifuge tube and 20 mL 80% ethanol was pipetted into each tube for the first extraction. The sealed tubes were put into an ultrasonic processor for 30 min at room temperature (Yong-Jin Chao et al, (2006), Ultrasonication-assisted extraction of resveratrol from grapes, Journal of Food Engineering 77: 725-730). The supernatants were separated from the pellet after centrifugation at 3000×g for 10 min. Ten mL of 80% ethanol was added to the pellet and ultrasonication was repeated followed by centrifugation as above. Two supernatants were pooled into a weighted centrifuge tube and evaporated to dryness by a Speedvac™ at room temperature. The dried extracts were weighed and the percent yields were calculated.

HPLC-MSD Analysis: The HPLC-APCI-MS method for the analyses was modified from Saleem et al. (Saleem A, Brendan R W, Harris C H, Muhammad A, Tayamo C, Sit S, Arnason J T. A validated method of phytochemical analysis of Flor-Essence™—A multiherb product. 2008. Phytochem Analysis Volume 20, Issue 5, pages 395-401). Briefly, an Agilent 1100™ Series HPLC-APCI-MSD system was used consisting of an autosampler, a quaternary pump, a column thermostat and a diode array detector. The mass spectrometric system, connected with HPLC system, was equipped with an atmospheric pressure chemical ionization (APCI) source, and a detector (range 50-1500 amu). All solvents were HPLC grade (Fisher Scientific, Calif., USA) unless otherwise specified. For analytical scale HPLC-MS analysis the solvents were sonicated for 5 min and the column (YMC-ODS AM-C18™, 100×2.0 mm, particle size 3.0 micron, Waters Inc., CA, USA) was equilibrated for 15 min with the starting conditions prior to analysis. The elution solvent system consisted of A=acetonitrile and B=trifluoroacetic acid 0.05% (aqueous), pH 3.5. The optimal gradient elution conditions were 5-100% TFA over 20 min. The column was subsequently washed by increasing B up to 100% over 5 min and kept at isocratic condition for 5 min. The column was brought back to initial conditions in 5 min and equilibrated for 3 min. The total run time was 30 min. The column was operated at a flow of 0.25 ml/min and its temperature was maintained at 48 ° C. with maximum pressure limit of 200 bars.

The extracts were reconstituted in 1 mg/ml in MeOH and filtered through 0.22 μm PTFE membranes and 10 μL of each extract were injected into the HPLC system though an autosampler. The rest of the filtrate was stored at 4° C. as a reference sample. The UV spectra were stored at monitoring wavelengths of 280 nm. The mass spectrometer was operated in scan mode using positive polarity with mass detection range of 100-800 amu. The optimized conditions were, fragmentor voltage 80, gain 1.0 EMV, nitrogen gas temperature 150 ° C./350 ° C. in negative ionization mode, vaporizer 420° C., drying gas flow 5.0 I/min, nebulizer pressure 60 psig, capillary voltage 2100 V/4000V in negative ionization mode and corona at 25 μA/5 μA in negative ion mode. The quantification of the identified markers in the extracts was performed by calibrating with known concentration of standards on the basis of area under the peaks using diode array detection.

### Results

Plant Extraction: The extraction yields, using the ultrasonication-assisted extraction method, ranged from 8.7% in M03 to 20.2% in E01 (Table 3).

### LC-MS Analysis

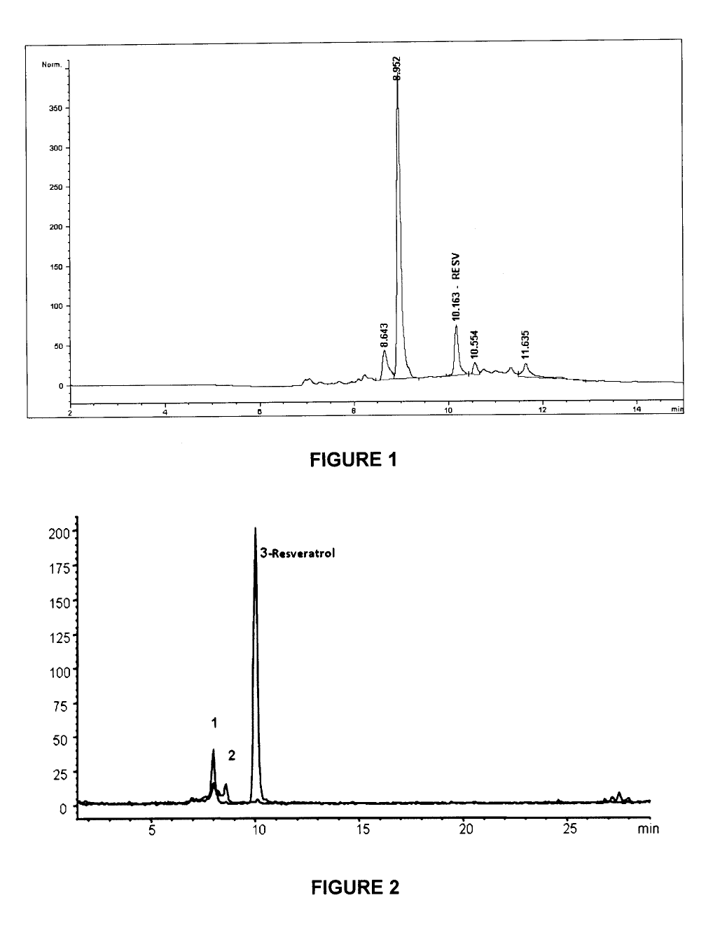

Identification of resveratrol: The confirmation of presence of resveratrol was achieved by HPLC-DAD and LC-MSD analyses. The identification of resveratrol in plant extract chromatograms (FIG. 1) was confirmed by performing a UV spectral match of the peak eluting at similar retention times with that of standard. Table 1 indicates the UV spectrometric characteristics of resveratrol obtained. The confirmation of the identification was further established by comparing the mass spectrometric fragmentation pattern of the standard resveratrol with the unknown peaks of the extracts eluting at the same time (FIG. 2). The two glycosides (1 and 2) were tentatively identified by MS (Table 2) eluting earlier than resveratrol. The chemical structure of these two resveratrol derivatives was not established.

TABLE 1
The identification of resveratrol and its derivatives by HPLC-MSD

| # | Compound | Rt (min) | UV (280 nm) | MSD [M + H]⁺ |
|---|---|---|---|---|
| 1 | Resveratrol | 9.1 | 210 sh, 300, 310 sh | 229 [M + H]⁺, |
| | glycoside-1 | | | 391 [M + glyc]⁺ |
| 2 | Resveratrol | 9.5 | 210 sh, 300, 310 sh | 229 [M + H]⁺, |
| | glycoside-2 | | | 391 [M + glyc]⁺ |
| 3* | Resveratrol | 10.1 | 210 sh, 300, 310 sh | 229 [M + H]⁺ |

*confirmed by a reference standard

TABLE 2
Resveratrol and its derivatives in plant extracts determined by HPLC-MSD

| | COMPOUND | | |
|---|---|---|---|
| | (see FIG. 2) | | |
| Plant extract | Glycoside 1 | Glycoside 2 | Resveratrol |
| E01 | | | ✓ |
| E08 | ✓ | ✓ | ✓ |
| E21 | | | ✓ |
| E46 | | | ✓ |
| E47 | ✓ | ✓ | ✓ |
| M02 | ✓ | | ✓ |
| M03 | | | ✓ |
| M23 | | | ✓ |

Quantification of resveratrol: A standard curve was generated by injecting 1 μL of five known concentrations of standard resveratrol, ranging from 500 μg/ml to 10 μg/ml dissolved in 100% methanol. As shown in Table 3 resveratrol is a predominant phytochemical and ubiquitously present in all extracts. The highest amount is found in E08 and M02 while it was detected in relatively lower amounts in E46, E47 and M21.

TABLE 3
Resveratrol content in selected plant extracts
as determined by HPLC-DAD.

| Plant | | Resveratrol content in dried |
|---|---|---|
| Extracts | Yield (%)* | plant material (μg/g)** |
| E01 | 20.2 | 258.6 ± 5.07 |
| E08 | 16.5 | 2480 ± 185. |
| E46 | 15.2 | 67.0 ± 0.57 |
| E47 | 13.8 | 1442. ± 31.2 |
| M02 | 14.2 | 455.4 ± 1.83 |
| M03 | 8.7 | 133.8 ± 0.55 |
| M21 | 19.3 | 108.5 ± 10.2 |
| M23 | 15.5 | 187.4 ± 1.17 |

*The plant extracts were obtained after drying the supernatants to dryness by a rotary evaporator for 30-45 min. The water bath temperature was set at 40 degree Celsius. Yield was calculated as (weight of dried extract/weight of dried plant material) × 100
**The values are average values ± stdev, n = 3 and n = 5 for E01 and E08

### Example 2

### Identification and Quantification of Resveratrol by HPLC in G. africanum Leaf and Stem Methanol Extracts

### Experimental

Stems and leaves from Gnetum africanum were received in dried unprocessed form and submitted to a methanol extraction. Gnetum africanum methanol extracts were separated using Lichrosphere 5RP-18™ column from Phenomax™ using mobile phase consisted of water (A) and acetonitrile (B), both containing 0.1% acetic acid which were applied in gradient elution.

### Results

Under the experimental HPLC conditions which were selected, a standard sample comprising a 1.0 μg of resveratrol had a retention time of 39 min. A resveratrol peak having a retention time 39 min was also identified within the 2 mg of leaf extract. Spiking the 2 mg of leaf extract with 1.0 μg of resveratrol increased the size of the resveratrol peak at 39 min. The spiking confirmed peak identity and provided an estimate for the quantity of resveratrol within this leaf extract.

It was calculated that about 1 μg resveratrol was present in the 2 mg of leaf extract (i.e. 0.5 μg/mg of dried leaves or 500 μg/g of dried leaves). Similarly, a resveratrol peak with a retention time 39 minutes was identified within the 2 mg of stem extract and it was calculated that this 2 mg stem extract contained about 33 μg of resveratrol (i.e. 16.5 pg/mg of dried stem or 16 500 μg/g of dried stem).

### Example 3

### Quantification of Biochemicals in Selected Parts of Gnetum africanum

Leaves and liana (stem) from Gnetum africanum growing either in Mayombe forest or the equatorial forest of Congo were analyzed for their biochemical content. A summary of the results are shown in Table 4.

TABLE 4
Biochemical analysis of Gnetum africanum

| | Forest of Mayombe | | Equatorial Forest | |
|---|---|---|---|---|
| | Leaves | Liana | Leaves | Liana |
| Protein content | 14.6% | 11.3% | 15.6% | 9.37% |
| Oil content | 3.2% | 3.1% | 6.4% | 1.6% |
| Omega 6 | 9.4% | 17.8% | | |
| Total trans fat | 29.8% | 39.4% | | |
| Cis fat | 12.4% | 21.1 | | |
| Omega 3 | 48.4 | 21.7 | | |
| Minerals (mg/100 g) | | | | |
| Bore (B) | 1.07 | 0.485 | N/A | N/A |
| Calcium (Ca) | 0.75 | 0.19 | 1.23 | 0.79 |
| Chrome (Cr) | 0.06 | 0.025 | N/A | N/A |
| Cobalt (Co) | 0.01 | 0.015 | N/A | N/A |
| Magnesium (Mg) | 43 | 32.49 | N/A | N/A |
| Manganese (Mn) | 12.15 | 4.58 | N/A | N/A |
| Iron (Fe) | 2.37 | 1.47 | N/A | N/A |
| Copper (Cu) | 0.63 | 1 | N/A | N/A |
| Nickel (Ni) | 0.055 | 0.05 | N/A | N/A |
| Phosphorus (P) | 44 | 49 | N/A | N/A |
| Potassium (K) | 1.45 | 1.44 | 0.62 | 1.02 |
| Selenium (Se) | 50.49 | 49.15 | N/A | N/A |
| Sodium (Na) | 0.5 | 0.055 | 0.018 | 0.030 |
| Zinc (Zn) | 0.41 | 0.66 | N/A | N/A |
| Lithium (Li) | 1.8 | 3.7 | N/A | N/A |
| Aluminium (Al) | 10.61 | 17.2 | N/A | N/A |
| Silicium (Si) | 8 | 3.76 | N/A | N/A |
| Titane (Ti) | 0.21 | 0.145 | N/A | N/A |
| Strontium (Sr) | 0.88 | 1.31 | N/A | N/A |
| Antimoine (Sb) | 0.14 | 0.01 | N/A | N/A |
| Rubidium (Rb) | 1.4 | 2.56 | N/A | N/A |
| Total fibers | 66.5 | 81.9 | 48.6 | N/A |
| Soluble fibers | 0.8 | 0.1 | 10.7 | N/A |
| Insoluble fibers | 65.7 | 81.1 | 37.9 | N/A |
| Resveratrol (mg/100 g) | 20.8 | 18.2 | 15.8 | 250 |
| Total sugar | 5.8% | 2.0% | 6.5% | 6.7% |
| Vitamin E mg/100 g | 71.8 | 1.8 | 38.2 | <1 |

Headings are included herein for reference and to aid in locating certain sections These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a compound” includes one or more of such compounds, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A processed plant material, wherein said processed plant material is dried and comprises at least about 30 μg resveratrol per gram of dried plant material.

2. The processed plant material of claim 1, wherein said dried plant material is derived from the plant Gnetum africanum.

3. (canceled)

4. The processed plant material of claim 2, wherein said dried plant material is derived from leaves or from a stem of the plant Gnetum africanum.

5. (canceled)

6. The processed plant material according to claim 1, wherein said processed plant material further comprises materials selected from the group consisting of resveratrol glycosides, lipids, minerals, fibers, sugars, vitamins, amino acids and proteins.

7. The processed plant material according to claim 1, wherein said processed plant material is packaged in a sealed container, a pouch, a jar, a tube, a blister pack, or a box.

8. A plant extract comprising at least about 0.005% w/w resveratrol.

9. The plant extract of claim 8, wherein said plant extract derives from the plant Gnetum africanum.

10. (canceled)

11. The plant extract of claim 9, wherein said plant extract derives from leaves or from a stem of the plant Gnetum africanum.

12. (canceled)

13. A method for obtaining a plant extract concentrated in resveratrol, comprising:
(i) providing plant material from Gnetum africanum;
(ii) submitting said plant material to a solvent extraction; and
(iii) obtaining a plant extract concentrated in resveratrol, said plant extract comprising at least about 0,005% w/w resveratrol.

14. The method of claim 13, wherein 1 g of dry plant material yield at least about 50 μg resveratrol.

15. The method of claim 13, wherein said solvent is selected from the group consisting of methanol, ethanol, ethyl acetate and combinations thereof.

16. The method of claim 13, wherein said plant material is derived from leaves and/or from a stem of Gnetum africanum.

17. The method of claim 13, wherein said plant extract further comprises a resveratrol glycoside selected from: (i) a compound identifiable under detection conditions of Table 1 by the presence of a peak having a retention time of 9.1 min on a HPLC-APCI-MSD chromatogram; and (ii) a compound identifiable under detection conditions of Table 1 by the presence of a peak having a retention time of 9.5 min on a HPLC-APCI-MSD chromatogram. 18-21. (Cancelled)

22. An edible product, comprising at least one of the following constituents:
(a) a dried processed plant material comprising at least about 30 μg resveratrol per gram of dried plant material;
(b) a plant extract comprising at least about 0.005% w/w resveratrol;
(c) a resveratrol glycoside selected from: (i) a compound identifiable under detection conditions of Table 1 by the presence of a peak having a retention time of 9.1 min on a HPLC-APCI-MSD chromatogram; and (ii) a compound identifiable under detection conditions of Table 1 by the presence of a peak having a retention time of 9.5 min on a HPLC-APCI-MSD chromatogram.

23. The edible product of claim 22, wherein said edible product is a food product selected from the group consisting of a dairy product, a nutritional bar, a herbal tea, and a drink.

24. (canceled)

25. The edible product of claim 22, wherein said edible product is a nutraceutical product is selected from the group consisting of vitamins, minerals, nutritional supplements, herbal remedies, homeopathic remedies, probiotics, amino acids, and essential fatty acids.

26. A cosmetic or pharmaceutical composition comprising at least one of the following constituents:
(a) a dried processed plant material comprising at least about 30 μg resveratrol per gram of dried plant material;
(b) a plant extract comprising at least about 0.005% w/w resveratrol;
(c) a resveratrol glycoside selected from: (i) a compound identifiable under detection conditions of Table 1 by the presence of a peak having a retention time of 9.1 min on a HPLC-APCI-MSD chromatogram; and (ii) a compound identifiable under detection conditions of Table 1 by the presence of a peak having a retention time of 9.5 min on a HPLC-APCI-MSD chromatograms;
in combination with a suitable cosmetic or pharmaceutically acceptable carrier, diluent or excipient.

27. The composition of claim 26, wherein said composition is a cosmetic composition is-selected from the group consisting of facial creams, lotions, and skin tonics.

28-29. (canceled)

30. The composition of claim 26, wherein said composition is a pharmaceutical composition is for the prevention or treatment of a disease selected from the group consisting of skin diseases, cardiovascular diseases, inflammatory diseases, blood lipids related conditions, and pain.

31-32. (canceled)

33. A method for the prevention or treatment of a disease selected from the group consisting of skin diseases, cardiovascular diseases, inflammatory diseases, blood lipids related conditions, and pain, comprising administering to a subject in need thereof at least one of the following constituents:
(a) a dried processed plant material comprising at least about 30 μg resveratrol per gram of dried plant material;
(b) a plant extract comprising at least about 0.005% w/w resveratrol;
(c) a resveratrol glycoside selected from: (i) a compound identifiable under detection conditions of Table I by the presence of a peak having a retention time of 9.1 min on a HPLC-APCI-MSD chromatogram; and (ii) a compound identifiable under detection conditions of Table 1 by the presence of a peak having a retention time of 9.5 min a HPLC-APCI-MSD chromatogram.

34. The processed plant material of claim 1, wherein said processed plant material is dried and comprises at least about 500 μg resveratrol per gram of dried plant material.

35. The processed plant material of claim 1, wherein said processed plant material is dried and comprises at least about 2500 μg resveratrol per gram of dried plant material.

36. The plant extract of claim 8, wherein said extract comprises at least about 0.01% w/w resveratrol.
